(19) United States
(12) Patent Application Publication
(10) Pub. No.: US 2010/0191336 A1
(43) Pub. Date: Jul. 29, 2010

(54) FIXATION DEVICE AND METHOD

(75) Inventor: E. Skott GREENHALGH, Lower Gwynedd, PA (US)

Correspondence Address: LEVINE BAGADE HAN LLP, 2400 GENG ROAD, SUITE 120, PALO ALTO, CA, 94303, US

(73) Assignee: Stout Medical Group. L.P., Perkasie, PA (US)

(21) Appl. No.: 12/694,221

(22) Filed: Jan. 26, 2010

Related U.S. Application Data
(63) Continuation-in-part of application No. 12/617,663, filed on Nov. 12, 2009.
Application No. 12/617,663 is a continuation-in-part of application No. 12/617,526, filed on Nov. 12, 2009.
(60) Provisional application No. 61/113,691, filed on Nov. 12, 2008.
(60) Provisional application No. 61/113,691, filed on Nov. 12, 2008.

(51) Int. Cl. A61F 2/44

(52) U.S. Cl. 623/17.16

(57) ABSTRACT

An implantable orthopedic stability device is disclosed. The device can have a contracted and an expanded configuration. A method of using the device between adjacent vertebral surfaces for support and/or fixation of either or both of the adjacent vertebrae is also disclosed.

### CROSS-REFERENCE TO RELATED APPLICATIONS

This application is a continuation-in-part of U.S. patent application Ser. No. 12/617,663, filed Nov. 12, 2009; and Ser. No. 12/617,526, filed Nov. 12, 2009, which claim the benefit of U.S. Patent Application No. 61/113,691, filed on Nov. 12, 2008.

### BACKGROUND OF THE INVENTION

1. Technical field

Devices and methods for fixation of tissue are disclosed. More specifically, the devices and methods can be for inter facet fusion of vertebrae or fusion of other bones to one another.

2. Background of the Art

A vertebroplasty device and method that eliminates or reduces the risks and complexity of the existing art is desired. A vertebroplasty device and method that may reduce or eliminate the need to inject a liquid directly into the compression fracture zone is also desired.

Other ailments of the spine result in degeneration of the spinal disc in the intervertebral space between the vertebral bodies. These include degenerative disc disease and traumatic injuries. In either case, disc degeneration can cause pain and other complications. Conservative treatment can include non-operative treatment requiring patients to adjust their lifestyles and submit to pain relievers and a level of underlying pain. Operative treatment options include disc removal. This can relieve pain in the short term, but also often increases the risk of long-term problems and can result in motor and sensory deficiencies resulting from the surgery. Disc removal and more generally disc degeneration disease are likely to lead to a need for surgical treatment in subsequent years. The fusion or fixation will minimize or substantially eliminate relative motion between the fixed or fused vertebrae. In surgical treatments, adjacent vertebra can be fixated or fused to each other using devices or bone grafts. These may include, for example, screw and rod systems, interbody spacers (e.g., PEEK spacers or allograft bone grafts) threaded fusion cages and the like.

Some fixation or fusion devices are attached to the vertebra from the posterior side. The device will protrude and result in additional length (i.e., needed to overlap the vertebrae) and additional hardware to separately attach to each vertebrae. Fusion cages and allografts are contained within the intervertebral space, but must be inserted into the intervertebral space in the same dimensions as desired to occupy the intervertebral space. This requires that an opening sufficient to allow the cage or graft must be created through surrounding tissue to permit the cage or graft to be inserted into the intervertebral space.

A spinal fixation or fusion device that can be implanted with or without the need for additional hardware is desired. Also desired is a fixation or fusion device that can be deployed in a configuration where overlapping the fixated or fused vertebrae is not required.

Also desired is an intervertebral device the may be inserted in to the intervertebral space at a first smaller dimension and deployed to a second, larger dimension to occupy the intervertebral space. The ability to insert an intervertebral spacer at a dimension smaller than the deployed dimension would permit less disruption of soft and boney tissue in order to access the intervertebral space.

### SUMMARY OF THE INVENTION

A device that can replace or supplement the screw or rod elements of a typical fusion system is disclosed. The device can be placed in the inter-facet space to fuse adjacent vertebrae and/or create a bone mass within the facet joint in a patient's spine. The device can be placed between adjacent vertebral bodies in the vetebral body articulating space, for example after a partial or complete discectomy in place of the removed disc.

The device can be less invasive than typical existing devices. For example, the device can be in a compacted (i.e., small) configuration when inserted into a patient and transformed into an expanded (i.e., large) configuration when positioned at the target site. For example, the device can be expanded when the device is between the inferior and superior facet surfaces. The device can create less soft tissue (e.g., bone) disruption than a typical fusion system. The device in an expanded configuration can improve anchoring within the joint, structural stability, and create an environment for bone healing and growth leading to fusion between adjacent vertebrae.

The device can have a first plate and a second plate. The device can be inserted and positioned into the joint so the first plate is in contact with a first articulating surface of the joint, and the second plate is in contact with a second articulating surface of the joint opposite of the first surface of the joint. For example, the opposite articulating surfaces can be opposed surfaces of vertebral plates or sides of a facet joint. Once inserted into the joint, the first plate can be rotatingly tilted away from the second plate and locked into position. The tilting and locking of the device can fuse the first articulating joint to the second articulating joint.

During deployment into tissue (e.g., bone), one, two or more holes can be drilled into the target site to create a deployment hole in which to insert the device. The deployment hole can be round or non-round (e.g., by drilling more than one overlapping or adjacent hole, or crafting a square or rectangular hole), for example to substantially match the transverse cross-section of the device in a contracted configuration.

The device can be cannulated, for example having a lateral (i.e., transverse or latitudinal) and/or lengthwise (i.e., longitudinal) channel through the device. The device can be deployed over a wire or leader, such as a guidewire. The device can be slid over the guidewire, with the guidewire passing through the longitudinal channel of the device.

### BRIEF DESCRIPTION OF THE DRAWINGS

FIG. 1a is a side perspective view of a variation of the device in a contracted configuration.

FIG. 1b is a variation of cross-section A-A of FIG. 1a.

FIG. 1c is a side perspective view of the device of FIG. 1a in an expanded configuration.

FIG. 1d is a variation of cross-section B-B of FIG. 1c.

FIG. 2a is side view of a variation of cross-section A-A of FIG. 1a.

FIG. 2b is side view of a variation of cross-section B-B of FIG. 1b.

FIG. 3a is a variation of cross-section A′-A′ of FIG. 1a.

FIG. 3b is a variation of cross-section B′-B′ of FIG. 1b.

FIG. 3c is a variation of FIG. 1a with the top plate absent.

FIG. 4 illustrates a variation of the device in a longitudinally expanded configuration.

FIG. 5 illustrates the device of FIG. 4 is a longitudinally contracted and radially expanded configuration.

FIG. 6 illustrates a visualization of a variation of a method for deploying the device into the spine between adjacent vertebrae.

FIGS. 7a and 7b illustrate visualizations of variations of the device deployed into the spine between adjacent vertebrae.

### DETAILED DESCRIPTION

A device 1 is disclosed that can be inserted into a target site 73 with the device 1 in a compressed or contracted (i.e., small) configuration. Once positioned in the deployment site, the device 1 can be transformed into an expanded (i.e., larger, bigger) configuration. The device 1 can be inserted and expanded in orthopedic target sites 73 for fixation and/or support. For example, the device 1 can be inserted and expanded over a guidewire between adjacent vertebral facet surfaces (i.e., within a facet joint 55).

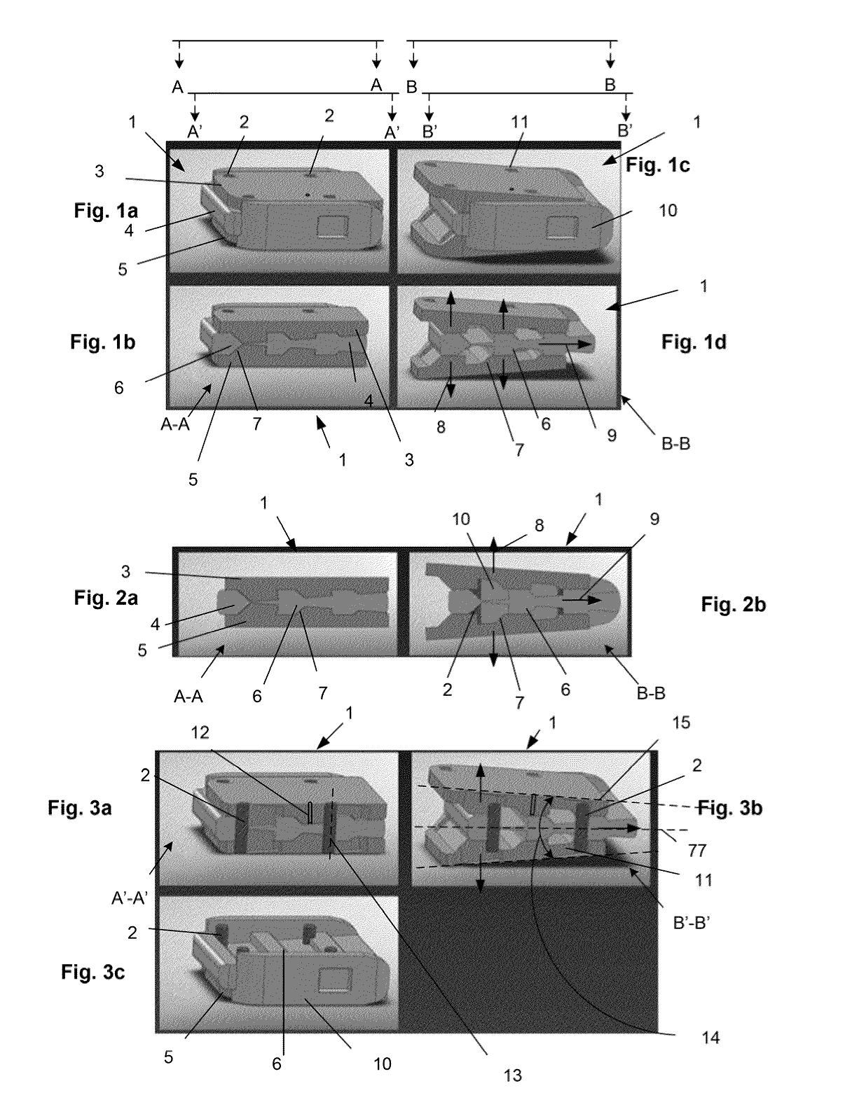

FIGS. 1a through 3c illustrate that the device 1 can have a top plate 3 attached to a bottom plate 5. The top plate 3 can be attached to the bottom plate 5 by one, two, three four or more pins 2. The plates can have a substantially flat external surface facing outward from the device 1. The pin longitudinal axes 13 can be substantially perpendicular to the plate surface planes of the external surfaces of the top 3 and bottom 5 plates when the device 1 is in a contracted configuration, and perpendicular to the device longitudinal axis 77.

The device 1 can have a middle plate 4 positioned between the top plate 3 and the bottom plate 5. The middle plate 4 can be slidably attached to the top plate 3 and the bottom plate 5. The pins 2 can be in pin slots 11 in the top 3 and/or bottom 5 and/or middle 4 plates. The pin slots 11 in the middle plate 4 can fix the pins 2 with respect to the position of the middle plate 4 in the direction of a device longitudinal axis 77. The pin slots 11 in the top 3 and bottom 5 plates can allow the pins 2 to move along a device longitudinal axis 77 with respect to the top 3 and bottom 5 plates to the extent of the pin slots 11, at which point the pin slots 11 will interference fit against the pins 2 to prevent further motion of the top 3 and bottom 5 plates. Accordingly, the top 3 and bottom 5 plates can slide with respect to each other and to the middle plate 4 in the direction of the device longitudinal axis 77 (and/or the middle plate 4 longitudinal axis).

The top plate 3 can have one or more angled and/or curved ramps 7 on the middle plate 4—side of the top plate 3. The bottom plate 5 can have one or more angled and/or curved ramped 7 on the middle plate 4—side of the bottom plate 5. The middle plate 4 can have angled and/or curved wedges 6 on the top plate 3—side and/or bottom plate 5—side of the middle plate 4. The wedges 6 can interface with the ramps 7. For example, the top 3 and bottom 5 plates can be in a contracted, compressed, or otherwise non-expanded configuration when the middle plate 4 is in a first position relative to the top 3 and bottom plates 5. The top and/or bottom 5 plates can be in an expanded, radially spread, or enlarged configuration when the middle plate 4 is in a second position (e.g., pulled away 9) relative to the top and/or bottom 5 plates.

The middle plate 4 can have no, one or two side walls 10. The side walls 10 can extend to about the height of the top plate 3 and/or bottom plate 5 when the device 1 is in a contracted or expanded configuration.

The top plate 3, bottom plate 5, side plates and combinations thereof can have ingrowth channels 12, windows, or ports. The ingrowth channels 12 can be configured to encourage bone growth into the ingrowth channel. For example, the ingrowth channels 12 can have textured surface and/or be coated and/or partially or completely filled with one or more osteogenic or osteoinductive material, for example any of those disclosed below.

FIGS. 3a and 3b illustrate that the pins 2 can be contained by the top 3 and bottom 5 plates during expansion 41 of the device 1. The pins 2 can be radiopaque and/or anti-torque. The side walls 10 can brace or otherwise interference fit the top and/or bottom 5 plates, for example to minimize lateral movement of the top and/or bottom 5 plates relative to the middle plate 4.

When the device 1 is in an expanded configuration, the top plate surface plane 15 and the bottom plate surface plane 29 can rotate away from each other, as shown by arrow 8, to form a device expansion angle 14. The device expansion angle 14 can be from about 1° to about 45°, more narrowly from about 2° to about 20°. For example, the device expansion angle 1° can be about 5° or about 10°. The device 1 can have a ratchet, or steps or teeth on the ramp 7 and wedges 6 to allow the device expansion angle 14 to be expanded at discrete increments, such as increased at increments of about 0.25°, about 0.5°, about 1°, or about 2°.

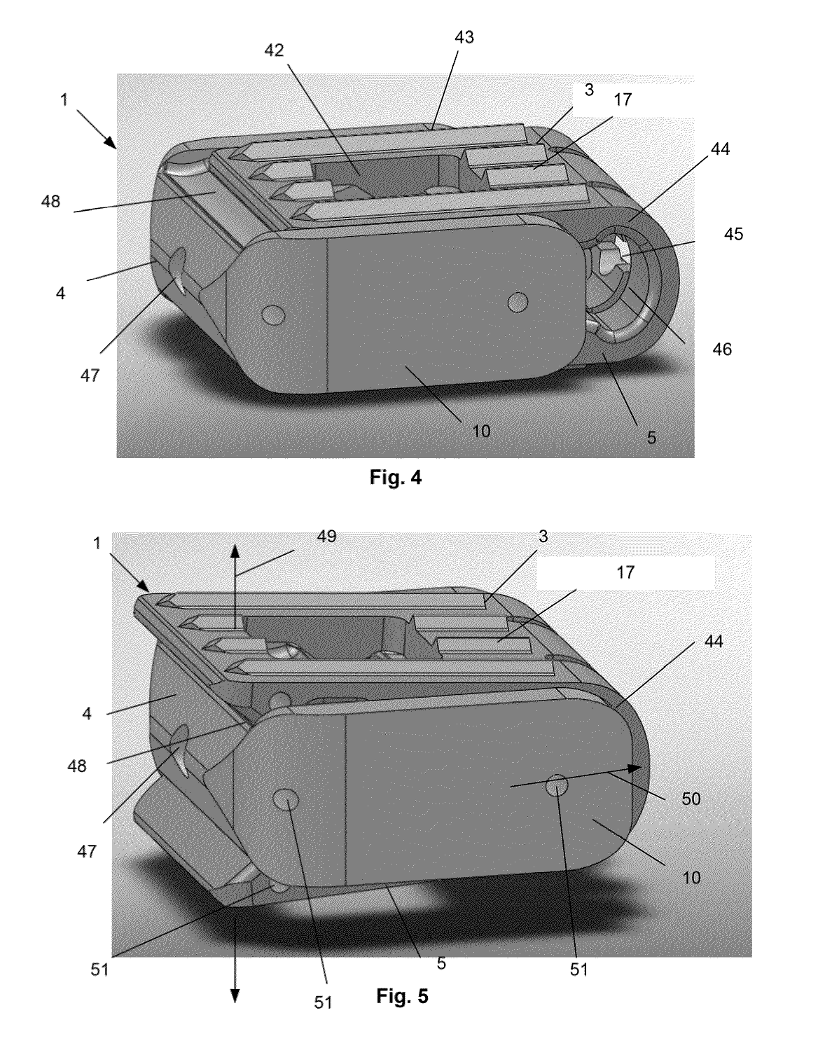

FIG. 4 illustrates that the top plate 3 can be rotatably attached to the bottom plate 5. The top plate 3 can be resiliently (i.e., elastically) attached to the bottom plate 5. The top plate 3 can be plastically defonnably (i.e., plastically) attached to the bottom plate 5. The top plate 3 and the bottom plate 5 can be integral with or attached to a plate hinge 44. The top plate 3 and bottom plate 5 can be attached at a first end at the plate hinge 44. The top plate 3 and bottom plate 5 can be unattached at a second end away from the plate hinge 44.

The top plate 3 and/or bottom plate 5 can have a surface texture 17 on the outward-facing surface. For example, the surface texture 17 can be ribs 43 oriented along the longitudinal axis of the device 1.

The top plate 3 and bottom plate 5 can form a side port 46. The middle plate 4 can be slidably received by the side port 46. The middle plate 4 can have a side wall 10. The side wall 10 can obstruct, cover, and/or seal the external side of the side port 46. The side port 46 can expand near the hinge 44. One or both side walls 10 can have inward-directed extensions that can snap-fit or otherwise engage into the expanded portions of the side ports 46. When the side wall 10 slides into the expanded portion of the side port 46, the side wall 10 can force the top plate 3 to rotate away from the bottom plate 5.

The middle plate 4 can have a middle plate port 47. The plate hinge 44 can have a plate hinge port 45. The middle plate port 47 and the plate hinge port 45 can be aligned along the longitudinal axis of the device 1. A deployment tool 35 can be releasably attached to the middle plate port 47 and/or the plate hinge port 45. The deployment tool 35 can compress the middle plate port 47 toward the plate hinge port 45.

The middle plate 4 can have one or more middle plate ramps 48, for example positioned adjacent to the inner surfaces of the top plate 3 and the bottom plate 5. When the middle plate 4 is longitudinally extended away from the top 3 and bottom 5 plates, as shown in FIG. 4, the plane of the top plate 3 can be can be substantially parallel to the plane of the bottom plate 5.

FIG. 5 illustrates that the middle plate 4 can be translated toward the plate hinge 44. For example, a deployment tool 35 can exert a compression force on the plate hinge 44 and the middle plate 4, translating the middle plate 4 toward the middle plate ramp 48, as shown by arrow 50. The top plate ramps can rotate, as shown by arrows 49, the top plate 3 away from the bottom plate 5.

The device 1 can have one or more radiopaque and/or echogenic markers 51. For example, the device 1 can have aligned markers 51 on the top plate 3, middle plate 4 and bottom plate 5. When the device 1 is in a contracted pre-deployment configuration, the markers 51 can be located immediately adjacent to one another, for example appearing as a single marker 51. When the device 1 is in an expanded configuration, the markers 51 can move apart from each other, indicating to a doctor performing the implantation and deployment procedure using visualization (e.g., x-ray or ultrasound-based) that the device 1 has expanded. Under visualization the markers 51 can also indicate the location and orientation of the device 1.

### Method of Using

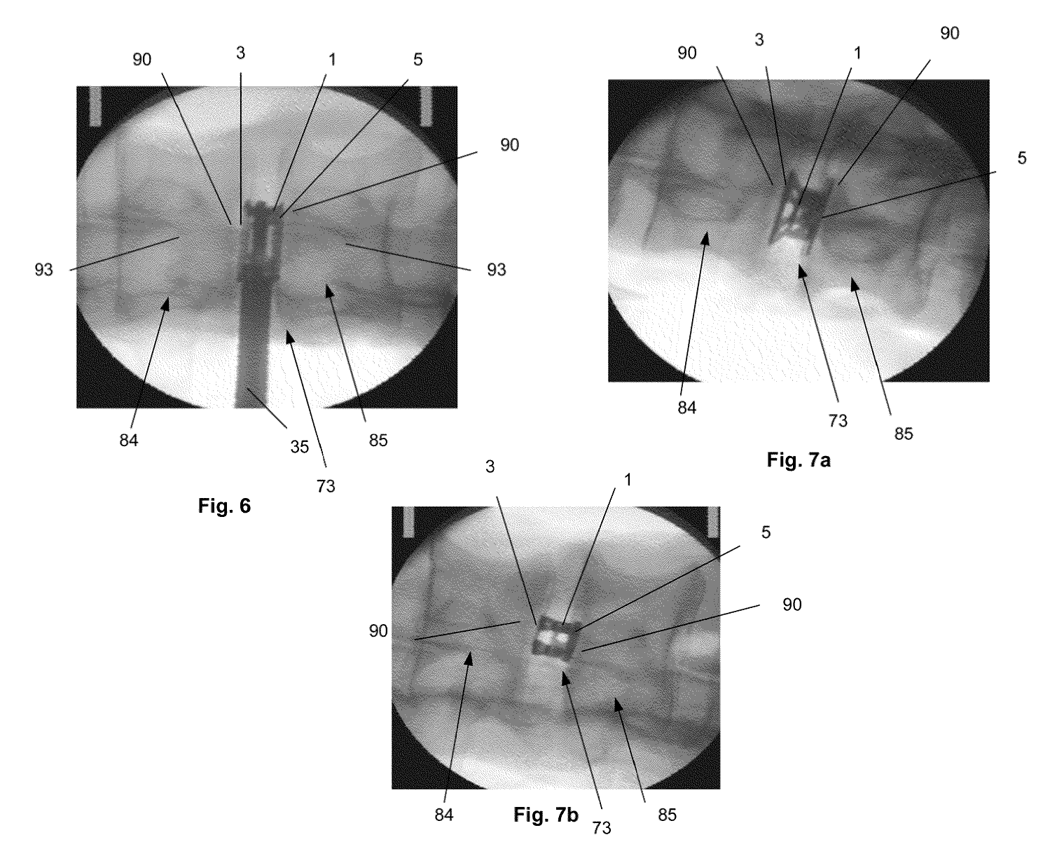

FIG. 6 illustrates the deployment tool 35 inserted to a target site 73 in vivo between a first vertebra 84 and a second vertebra 85. For example, the device 1 can be placed at the target site 73 after a partial or complete discectomy. When the device 1 is in a contracted configuration, the tool can position the device 1 between a first vertebral body 92 of the first vertebra 84 and a second vertebral body 93 of the second vertebra 85. The device 1 can be inserted into the target site 73 a direction substantially parallel to the surfaces of the vertebral body end plates. The device 1 can be placed between a first vertebral end plate 90 of the first vertebral body 92 and the adjacent second vertebral end plate 91 of the second vertebral body 93. In this inter-vertebral location, the top plate 3 of the device 1 can be in contact with or directly adjacent to the first vertebral end plate 90. The bottom plate 5 of the device 1 can be in contact with or directly adjacent to the second vertebral end plate 91.

FIGS. 7a and 7b illustrate that the deployment tool 35 can radially expand the device 1 between the first vertebral end plate 90 and the second vertebral end plate 91. The top plate 3 can press against and/or embed into the first vertebral end plate 90. The bottom plate 5 can press against and/or embed into the second vertebral end plate 91. The device 1 can fuse the first vertebra 84 to the second vertebra 85.

The device 1 can be filled with a filled before or after radial expansion. Tissue ingrowth can occur into the top plate 3 through the top ports 42, bottom plate 5 through the bottom ports, and elsewhere through the device 1.

Any or all elements of the device 1 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, Del.), poly ester amide (PEA), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

The device 1 can be made from substantially 100% PEEK, substantially 100% titanium or titanium alloy, or combinations thereof.

Any or all elements of the device 1 and/or other devices or apparatuses described herein, can be, have, and/or be completely or partially coated with agents for cell ingrowth.

The device 1 and/or elements of the device 1 and/or other devices or apparatuses described herein can be filled, coated, layered and/or otherwise made with and/or from cements, fillers 70, and/or glues known to one having ordinary skill in the art and/or a therapeutic and/or diagnostic agent. Any of these cements and/or fillers 70 and/or glues can be osteogenic and osteoinductive growth factors.

Examples of such cements and/or fillers 70 includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Spl Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

I claim:

1. A method of using an orthopedic support device comprising:
inserting the support device into an orthopedic target site;
longitudinally contracting the device;
rotationally expanding the device during the longitudinal contraction of the device.

2. The method of claim 1, wherein the device comprises a first plate and a second plate, and wherein the rotational expansion of the device comprises rotating the first plate with respect to the second plate.

3. The method of claim 2, wherein rotating the first plate with respect to the second plate comprises rotating the first plate with respect to the second plate about a rotational axis at the terminal end of the first plate.

4. The method of claim 2, wherein the inserting of the support device comprises placing the first plate adjacent to a first vertebral body, and placing the second plate adjacent to a second vertebral body, wherein the first vertebral body is in a first vertebra immediately adjacent to a second vertebral body comprising the second vertebral body.

5. The method of claim 2, wherein the first plate is integral with the second plate.

6. A method of using an orthopedic support device comprising:
inserting the support device into an orthopedic target site, wherein the orthopedic target site is in a space between a first vertebral body and a second vertebral body;
pushing the first vertebral body away from the second vertebral body, wherein the pushing comprises rotating the first vertebral body with respect to the second vertebral body, and wherein pushing comprises longitudinally contracting the support device.

7. The method of claim 6, further comprising rotationally expanding the device during the longitudinal contraction of the device.

8. An implantable orthopedic device having a longitudinal axis comprising:
a first plate,
a second plate, wherein a first end of the first plate is rotationally attached to a first end of the first plate, and
a third plate slidably interfacing between the first and second plates.

9. The device of claim 8, wherein the first plate is integral with the second plate.

10. The device of claim 9, wherein the first plate is resiliently integral with the second plate.

11. The device of claim 8, wherein the first plate is substantially parallel with the second plate when the device is in an unbiased configuration.

12. The device of claim 8, wherein a plane defined by the first plate forms an angle with a plane defined by the second plate, and wherein when the device is in a longitudinally contracted configuration, the angle is greater than about 5° and less than about 45°.

13. The device of claim 8, wherein the third plate comprises a wedge, and wherein the wedge is configured to expand a second end of the first plate away from the second end of the second plate.
